(19) United States
(12) Patent Application Publication
(10) Pub. No.: US 2016/0000734 A1
(43) Pub. Date: Jan. 7, 2016

(54) METHOD AND COMPOSITIONS OF CIVAMIDE TO TREAT DISEASE OF THE INTESTINES

(71) Applicant: WINSTON LABORATORIES, INC., Vernon Hills, IL (US)

(72) Inventor: Joel E. Bernstein, Deerfield, IL (US)

(21) Appl. No.: 14/853,200

(22) Filed: Sep. 14, 2015

Related U.S. Application Data
(63) Continuation of application No. 12/705,888, filed on Feb. 15, 2010, now abandoned.

(51) Int. Cl. A61K 31/165; A61K 9/48

(52) U.S. Cl. CPC A61K 31/165; A61K 9/4858

(57) ABSTRACT

Compositions and a methods are described for treating disorders of the small and large bowel, such as Crohn's disease, ulcerative colitis and irritable bowel syndrome, without producing any treatment-related systemic side effects and with minimal or no abdominal discomfort. A method of administration of civamide that is incorporated into soft gelatin capsules and administered with meals is described. Surprisingly, the method provides for diminishment of pain and inflammation of the small and large bowel without producing any systemic side effects or significant abdominal discomfort.

### CROSS REFERENCE TO RELATED APPLICATIONS

This application is a Continuation of copending U.S. application Ser. No. 12/705,888, filed Feb. 15, 2010.

### BACKGROUND

Methods and compositions of civamide to treat diseases of the small and large intestines are described.

Civamide (cis-8-methyl-N-vanillyl-6-nonenamide), also known as zucapsaicin, is a stereoisomer of the chemical capsaicin which has been utilized over the last three decades to study a variety of neurophysiological processes. Civamide was previously found to be useful in the treatment of painful, inflammatory or allergic disorders, and was effective in such disorders, yet with significantly less of the localized burning and stinging associated with capsaicin's use.

Compositions containing civamide were reported as comparable in efficacy to compositions containing capsaicin, but with significantly less local adverse effects normally associated with capsaicin.

Compositions for oral administration of civamide and a method of treating painful or allergic disorders by oral administration of such compositions were based on pharmacokinetic (“PK”) and analgesic studies in mice, rats, and dogs. These studies demonstrated that civamide was reasonably well absorbed systemically in rodents and dogs (approximately 8-12% absorption of an administered oral dose) and that these blood levels were accompanied by effective analgesia as demonstrated by the Chung, Formalin and hot-plate animal models.

### SUMMARY

Compositions and a methods are described for treating disorders of the small and large bowel, such as Crohn's disease, ulcerative colitis and irritable bowel syndrome, without producing any treatment-related systemic side effects and with minimal or no abdominal discomfort.

A method of administration of civamide that is incorporated into soft gelatin capsules and administered with meals is described. Surprisingly, the method provides for diminishment of pain and inflammation of the small and large bowel without producing any systemic side effects or significant abdominal discomfort.

### DETAILED DESCRIPTION

Based on pharmaceutical and analgesic studies in rodents and dogs, a soft gel capsule containing civamide was developed as a potential treatment for neuropathic and/or postoperative pain. However, when these capsules were administered orally to normal human volunteers, two unexpected results occurred:

(1) Utilizing a very sensitive analytical procedure (detecting civamide serum levels to 0.01 ng/ml), NO civamide was found to be absorbed by serum; and

(2) Civamide produced more local burning and discomfort in the gastrointestinal tract than comparable dosages of capsaicin.

Taking into account these results, it was surmised that diseases of the bowel in human patients might be amenable to treatment with civamide without producing any systemic absorption or systemic side effects. Because the soft gel capsules administered orally produced an unacceptable degree of pain and discomfort, to rectify this problem, the soft gel capsules were coated with an enteric coating. Utilizing an enteric coating, bypassed digestion of the capsule in the stomach resulting in the first release of civamide in the small bowel and reduced abdominal pain and discomfort.

The pharmacokinetic (PK) study was repeated in normal human volunteers with the enteric coated capsule. Once again, there was no systemic absorption of civamide, and abdominal discomfort was reduced although not eliminated. The PK study was repeated with administration of the enteric coated capsules with a meal, and the results again were no systemic absorption of civamide and abdominal discomfort was essentially non-existent.

Formulations are provided for use with the method that incorporate civamide into soft gelatin capsules which may be enteric coated to prevent dissolution by acid in the stomach. In each of the foregoing formulations, civamide may be present in a single dosage of from about 0.5 mg-100 mg, suitable for oral administration to patients. The civamide can be present as the compound civamide or as pharmaceutically acceptable salt thereof, such as a hydrochloride salt or an acetate salt. Civamide was supplied by Winston Laboratories, Vernon Hills, Ill.

The method includes administering compositions containing civamide orally in pharmaceutically acceptable vehicles in order to provide pain and inflammation diminishment in the small or large bowel. Conditions amenable to such treatment include Crohn's disease, ulcerative colitis and irritable bowel syndrome. Civamide or a salt of civamide are present in each dose in the amount of about 0.5 mg to about 100 mg (weight/weight).

### EXAMPLES

Examples are provided for illustrative purposes and are not intended to limit the scope of the disclosure.

### Example 1

Civamide soft gel capsules were prepared by mixing 0.5 mg, 5 mg, 20 mg and 100 mg of civamide dissolved in a lipophilic vehicle containing inactive ingredients: Mono/Diglycerides of Capryl/Capric Acid (capmul MCM), Caprylocapryl Macrogol Glycerides (Labrasol), and Polysorbate 80, triglycerides, and encapsulating the resultant mixture into capsules made of gelatin acid hide (Type 195) sorbital special blend.

### Example 2

Capsules of Example 1 were enteric coating with a mixture of Methacrylic Acid Copolymer, Dispersion (Eudragit L30 D-55); PEG 400; Talc; Simethicone Emulsion and Purified Water, by methods well known to those of skill in the art.

### Example 3

Capsules described in Example 1, were administered to six (6) normal volunteers, bloods were drawn and serum concentrations of civamide were measured at 5, 30, 60, 90, 120, 150, 180, 240 and 360 minutes after administration of the capsules. None of the subjects had any measurable (at an assay sensitivity of 0.01 ng/ml) serum civamide at any measuring point in the study. However, subjects complained of severe abdominal distress that lasted from less than 30 minutes up to several hours following administration of the drug.

### Example 4

Enteric capsules as described in Example 2 above were administered to six (6) normal volunteers on an empty stomach and one week later immediately following a meal. In both cases there were no measurable serum levels of civamide at any time point in the study (from 5 to 360 minutes after drug administration). Abdominal distress was substantially less than with the capsules in Example 1 and 3 (i.e. generally mild) following ingestion of the enteric coated capsules on an empty stomach. Abdominal distress was virtually nonexistent in subjects who ingested the enteric coated capsules of Example 2 after eating.

### Example 5

For four weeks patients with Crohn's disease and ulcerative colitis are administered enteric coated 2.5 mg, 5 mg and 20 mg civamide capsules twice daily following eating. At the end of the four-week treatment period, most patients will demonstrate a significant decrease in their crampy abdominal pain and diarrhea. The phase II design consists of:
(a) 45 patients with Crohn's disease activity index (CDAI) ≧220 to ≦450;
(b) concomitant stable doses of aminosalicylates, corticosteroids, and/or immunomodulatory agents are permitted;
(c) there are 3 drug groups—2.5 mg civamide BID, 5 mg civamide BID, and placebo capsules BID;
(d) there is a 4-week treatment period and a 2-week post-treatment observation period; and
(e) clinical remission (defined as CDAI <150) evaluated at end of 4-week treatment period and following 2-week post-treatment observation period

### Example 6

Patients with irritable bowel syndrome with episodic crampy abdominal pain, alternating constipation and diarrhea and abdominal distention are administered 20 mg enteric coated civamide soft gel capsules once daily after breakfast for 4 weeks. Patients will have less abdominal pain and distention as well as more normal bowel movements by the study's end.

### MATERIALS AND METHODS

### Enteric Coated Capsules

Capsule and oral formulations particularly useful to practice the claimed methods include those selected from the group consisting of instant release capsules, sustained release capsules, delayed release capsules that are enteric coated. In an embodiment, a capsule dosage form is an enteric coated rapid onset capsule dosage form.

A sustained release dosage form may include a core coated with an enteric coating so that the core does not dissolve in the stomach. The core may be either a sustained release core, e.g., a matrix tablet or an osmotic tablet, or alternatively may be an immediate release core that provides a delayed burst. By “enteric coating” is meant an acid resistant coating that remains substantially intact and does not substantially dissolve at a pH of less than about 4. The enteric coating surrounds the core so that the core does not dissolve in the stomach. The enteric coating may include an enteric coating polymer.

Examples of components used for enteric coating include cellulose acetate phthalate (CAP), methyl acrylate-methacrylic acid copolymers, cellulose acetate succinate, hydroxy propyl methyl cellulose phthalate, hydroxy propyl methyl cellulose acetate succinate (hypromellose acetate succinate), polyvinyl acetate phthalate (PVAP), methyl methacrylate-methacrylic acid copolymers, sodium alginate and stearic acid.

In an embodiment, the enteric coating polymers are generally polyacids having a pKₐ of about 3 to 5. Examples of enteric coating polymers include for example, cellulose derivatives, such as cellulose acetate phthalate, cellulose acetate trimellitate, hydroxypropyl methyl cellulose acetate succinate, cellulose acetate succinate, carboxy methyl ethyl cellulose, methylcellulose phthalate, and ethylhydroxy cellulose phthalate; vinyl polymers, such as polyvinyl acetate phthalate, polyvinylbutyrate acetate, vinyl acetate-maleic anhydride copolymer; polyacrylates; and polymethacrylates such as methyl acrylate-methacrylic acid copolymer, methacrylate-methacrylic acid-octyl acrylate copolymer; and styrene-maleic mono-ester copolymer. These components may be used either alone or in combination, or together with other polymers than those mentioned above as long as enteric coating is maintained.

In an embodiment, one class of preferred coating materials include the pharmaceutically acceptable methacrylic acid copolymer which are copolymers, anionic in character, based on methacrylic acid and methyl methacrylate, for example having a ratio of free carboxyl groups:methyl-esterified carboxyl groups of 1:>3, e.g. around 1:1 or 1:2, and with a mean molecular weight of 135000. Some of these polymers are known and sold as enteric polymers, for example having a solubility in aqueous media at pH 5.5 and above, and the commercially available enteric polymers.

The coating may include conventional plasticizers, including dibutyl phthalate; dibutyl sebacate; diethyl phthalate; dimethyl phthalate; triethyl citrate; benzyl benzoate; butyl and glycol esters of fatty acids; mineral oil; oleic acid; stearic acid; cetyl alcohol; stearyl alcohol; castor oil; corn oil; coconut oil; and camphor oil; and other excipients such as anti-tack agents, glidants, etc.

The enteric coating may also include insoluble materials, such as alkyl cellulose derivatives such as ethyl cellulose, crosslinked polymers such as styrene-divinylbenzene copolymer, polysaccharides having hydroxyl groups such as dextran, cellulose derivatives which are treated with bifunctional crosslinking agents such as epichlorohydrin, dichlorohydrin, 1,2-, 3,4-diepoxybutane, etc. The enteric coating may also include starch and/or dextrin.

I claim:

1. A method of providing systematic relief of a disorder selected from the group consisting of abdominal pain, abdominal distension, constipation and diarrhea, in patients with a condition selected from the group consisting of Crohn's disease and ulcerative colitis, the method comprising the oral administration of a pharmaceutical composition comprising an agent from the group consisting of civamide (cis-8-methyl-N-vanillyl-6-nonenamide) and one of its salts, in an amount of about 0.5 mg to 100 mg, incorporated into a lipophilic mixture and encapsulated in a soft gelatin capsule, whereby there is no systemic absorption of civamide or one of its salts.
